(19) United States
(12) Patent Application Publication
(10) Pub. No.: US 2022/0204923 A1
(43) Pub. Date: Jun. 30, 2022

(54) CHEMICALLY DEFINED MEDIUM FOR DIFFERENTIATION OF MUSCLE STEM CELLS IN VITRO

(71) Applicant: NANJING JOES FUTURE FOOD TECHNOLOGY CO., LTD., Nanjing (CN)

(72) Inventors: Guanghong ZHOU, Nanjing (CN); Zhongyuan WU, Nanjing (CN); Xinglian XU, Nanjing (CN); Shijie DING, Nanjing (CN); Huixia LI, Nanjing (CN)

(73) Assignee: NANJING JOES FUTURE FOOD TECHNOLOGY CO., LTD., Nanjing (CN)

(21) Appl. No.: 17/582,730

(22) Filed: Jan. 24, 2022

(30) Foreign Application Priority Data
Dec. 24, 2020 (CN) 202011558188.3

(51) Int. Cl. C12N 5/0735

(52) U.S. Cl. CPC C12N 5/0606; C12N 2501/115; C12N 2501/105; C12N 2500/38; C12N 2501/33; C12N 2500/36; C12N 2500/90; C12N 2501/39; C12N 2500/25

(57) ABSTRACT

A chemically defined medium for differentiation of muscle stem cells in vitro, namely a serum-free, more efficient and inexpensive chemically defined medium for inducing differentiation of muscle stem cells in vitro. Compared with an existing general muscle stem cell differentiation medium, using the chemically defined medium can increase the relative expression of myogenin genes by 4.48 times on the 2^{nd} day of differentiation, increase the relative expression of myosin heavy chain genes by 55.28 times on the 6^{th} day, and increase the percentage of cell differentiation from 34.94% to 57.93% in the terminal differentiation stage, and more, thicker and longer muscle fibers are formed through induced differentiation. The chemically defined differentiation medium further improves the differentiation efficiency of muscle stem cells, and provides a more efficient and inexpensive method for differentiation of muscle stem cells into myotubes, and for 3D culture of muscle stem cells to produce cell cultured meat.

### TECHNICAL FIELD

The present invention belongs to the technical field of stem cell and animal cell cultured meat, and in particular relates to a chemically defined medium for differentiation of muscle stem cells in vitro.

### BACKGROUND

Cell cultured meat is based on the growth and repair mechanism of animal muscles and is obtained by culturing animal muscle stem cells in vitro. Cell cultured meat does not require animal breeding and directly uses cells for industrialized production of meat. As a subversive way of meat production, cell cultured meat provides a new way to supplement meat protein supply in the future and realize green meat production. According to calculations, compared with traditional animal husbandry, the cell cultured meat industry can reduce energy consumption by 35% to 60%, occupy less land by 98%, and produce less greenhouse gases by 80% or more.

Muscle stem cells, also known as satellite cells, are located below the basilar membrane. Muscle stem cells originate from the mesoderm at the early stage of vertebrate embryogenesis, and are a necessary condition for skeletal muscle regeneration. After muscle injury, muscle stem cells are activated to enter a cell cycle and proliferate rapidly. Most of the cells differentiate and fuse to form muscle fibers, and a few cells renew themselves and restore a resting state to replenish a satellite cell pool. Similarly, a production process of cultured meat in vitro requires enrichment of seed cells to induce differentiation, thereby promoting fusion of most muscle stem cells to form muscle fibers. Therefore, choosing an excellent cell differentiation medium is a key link in a production process of cultured meat, and directly affects myotube fusion and protein production.

At present, a commonly used medium formula for induced differentiation of muscle stem cells in vitro is a Dulbecco's modified eagle medium (DMEM) basal medium with 2% horse serum and 1% double antibody added. The medium can support a basic cell differentiation process, and the process reaches the highest myotube fusion rate around the 5^{th} day of differentiation. However, due to addition of the horse serum, the chemical composition of the medium is not defined, and the medium has the problems of unstable composition of different batches of medium, easy contamination by pathogens such as viruses, high cost, etc. In addition, the differentiation percentage of muscle stem cells in the medium is only about 35%, and the differentiation efficiency is low. This means that the serum-containing medium cannot be used to produce cell cultured meat efficiently, stably and cheaply on a large scale, seriously hindering the progress of industrialization of cell cultured meat. Therefore, it is particularly important to develop a chemically defined serum-free differentiation medium capable of promoting efficient differentiation of muscle stem cells.

### SUMMARY

In view of the problems in the prior art, the objective of the present invention is to replace a serum component in a traditional differentiation medium and provide a chemically defined differentiation medium of muscle stem cells and an application method thereof.

The first objective of the present invention is to provide a chemically defined modified cell differentiation medium for differentiation of muscle stem cells in vitro, the modified cell differentiation medium being serum-free. The being serum-free refers to no addition of any animal serum components including horse serum, fetal bovine serum, bovine serum and human serum, and the modified cell differentiation medium is a muscle stem cell differentiation medium added with cell culture cofactors. By adding the cofactors, serum components in a traditional muscle stem cell differentiation medium are replaced.

Further, the muscle stem cell differentiation medium includes a muscle stem cell basal medium and a penicillin-streptomycin double antibody solution, and the volume ratio of the muscle stem cell basal medium to the penicillin-streptomycin double antibody solution is 99:1 (v/v).

Preferably, the muscle stem cell basal medium is one selected from DMEM, MEM, DMEM/F12, and F10.

Preferably, in the penicillin-streptomycin double antibody solution, the content of penicillin is 10000 U/ml, and the content of streptomycin is 10 mg/ml.

Further, the cell culture cofactors are several selected from the following cell culture supplement factors: N-2-hydroxyethylpiperazine-N-2-ethanesulfonic acid, PEG-PPG-PEG, serum albumin, Tween, transferrin, ethanolamine, cholesterol, vitamin E, palmitic acid, stearic acid, insulin, palmitoleic acid, linolenic acid, arachidonic acid, myristic acid, oleic acid, sodium selenite, linoleic acid, and insulin-like growth factor (IGF).

Further, in the modified cell differentiation medium, the total concentration of the cell culture cofactors added is in a range of 0.05-50 mg/mL.

Preferably, in the modified cell differentiation medium, the total concentration of the cell culture cofactors added is in a range of 0.5-30 mg/mL.

Further preferably, in the modified cell differentiation medium, the total concentration of the cell culture cofactors added is 3 mg/mL.

Further, in the modified cell differentiation medium, the concentration of any cell culture cofactor added is in a range of 0.5 ng/mL-100 mg/ml.

Preferably, in the modified cell differentiation medium, the concentration of any cell culture cofactor added is in a range of 50 ng/mL-50 mg/ml.

Further preferably, in the modified cell differentiation medium, the concentration of any cell culture cofactor added is in a range of 50 ng/mL-1.2 mg/ml.

The second objective of the present invention is to provide an application of the aforementioned chemically defined modified cell differentiation medium for differentiation of muscle stem cells in vitro to induction of differentiation of muscle stem cells in vitro, and specifically, the aforementioned modified cell differentiation medium is used to induce differentiation of muscle stem cells in vitro.

Further, the modified cell differentiation medium can promote differentiation of muscle stem cells in a process of inducing differentiation in vitro. Specifically, the modified cell differentiation medium can improve the differentiation efficiency, differentiation ability, and differentiation level of muscle stem cells in the process of inducing differentiation in vitro.

Further, the modified cell differentiation medium can increase the expressions of myogenin (MYOG), myosin heavy chain (MYHC) and caveolin-3 (CAV-3) genes in the process of inducing differentiation of muscle stem cells in vitro.

Further, the modified cell differentiation medium can increase synthesis of MYHC in the differentiation process of muscle stem cells in vitro.

As a special implementation of the application, a method for inducing differentiation of muscle stem cells in vitro using the aforementioned chemically defined modified cell differentiation medium for the differentiation of muscle stem cells in vitro includes the following steps:

1) taking purified primary muscle stem cells;

2) proliferating the muscle stem cells to a density of 90% or more in a culture dish plated with Matrigel using a muscle stem cell proliferation medium;

3) after blotting the muscle stem cell proliferation medium, adding the aforementioned modified cell differentiation medium to rinse the cells, blotting the medium again, replenishing with the modified cell differentiation medium, and continuing culture to induce differentiation; and

4) performing half medium change with the aforementioned modified cell differentiation medium every 2 days, and continuing culture until the cells fuse to form muscle fibers.

After induction of differentiation of the muscle stem cells in vitro for 5 days using the modified cell differentiation medium as described in the present invention, 57.93% of the cells fuse to form muscle fibers.

The purified cells in step 1) refer to muscle stem cells isolated from young pigs, and the muscle stem cells have a Pax7 surface antibody positive rate of 90% or more after being sorted by a flow cytometer.

Further, the aforementioned cells are cultured in a CO₂ incubator at 37° C., and the CO₂ concentration in a CO₂ incubator is 5% (v/v).

Further, the Matrigel plating concentration in step 2) is Matrigel: PBS=1:50 (Val). The muscle stem cell proliferation medium used contains 20 vol % of fetal bovine serum, 79 vol % of F10, 1 vol % of penicillin-streptomycin double antibody, and 1-10 ng/ml fibroblast growth factors.

The third objective of the present invention is to provide an application of the aforementioned modified cell differentiation medium in preparation of cultured meat, and the application includes the following steps:

1) mixing type I collagen, a DMEM containing phenol red, and a sodium hydroxide solution to obtain a mixed solution;

2) mixing muscle-derived cells with the mixed solution obtained in step 1) to obtain a cell-containing mixed solution; and

3) adding the cell-containing mixed solution obtained in step 2) to a porous reticular production mold of cultured meat for performing culture; performing culture in a 5% carbon dioxide incubator at 37° C. for 2 h to form hydrogel muscle tissues; after adding a proliferation medium to the hydrogel muscle tissues for 1 day, replacing the proliferation medium with the aforementioned chemically defined modified cell differentiation medium; culturing the hydrogel muscle tissues in the medium, and performing culture in the porous reticular production mold of cultured meat for 5 days to obtain porous reticular muscle tissues.

Further, the volume ratio of the type I collagen to the DMEM containing phenol red is 50:40, and the pH value is adjusted to 7.3-7.5 by adding the sodium hydroxide solution.

Further, step 1) includes adding Matrigel to the mixed solution, the volume ratio of the Matrigel to the mixed solution is 8:91.5, the Matrigel and the mixed solution are uniformly mixed and added to the mold for culture, and the Matrigel can help the reticular muscle tissue differentiation.

Further, the muscle-derived cells in step 2) are one of muscle stem cells, muscle progenitor cells, and muscle precursor cells, and in the cell-containing mixed solution, the density of the muscle-derived cells is 1×10⁵ cells/ml−1×10⁷ cells/ml.

Further, the porous reticular production mold of cultured meat is a porous reticular production mold of cultured meat as described in the patent with the number ZL201921875316.X.

The fourth objective of the present invention is to provide cultured meat obtained by the aforementioned modified cell differentiation medium.

Compared with use of a conventional differentiation medium to induce differentiation, using the modified cell differentiation medium to induce differentiation of the muscle stem cells inoculated in the porous reticular production mold of cultured meat can improve expression of myosin heavy chains, a differentiation marker of muscle stem cells.

### Beneficial Effects Implemented by the Technical Solution of the Present Invention are as Follows

A key link of production of cell cultured meat is that muscle stem cells enter a differentiation process, and the cells fuse with each other to form muscle fibers. However, the existing muscle stem cell differentiation medium has the disadvantages of undefined chemical composition, high cost, etc., and the applications in biochemistry, medicine, food science and the like are limited. Moreover, the induced differentiation efficiency of muscle stem cells is low, and the differentiation efficiency of cells after induction of differentiation for 5 days in the differentiation medium is only 35.94%, thus the differentiation potential of the muscle stem cells cannot be fully utilized. This means that the serum-containing medium cannot be used to produce cell cultured meat efficiently, stably and cheaply on a large scale, seriously hindering the progress of industrialization of cell cultured meat.

The chemically defined modified cell differentiation medium used for differentiation of muscle stem cells in vitro in the present invention replaces serum in the differentiation medium by adding cell culture cofactors, and avoids the problems of undefined serum chemical composition, unstable medium composition in different batches, easy contamination by pathogens such as viruses, high cost, etc. Also, using the modified cell differentiation medium provided by the present invention, in inducing the differentiation of muscle stem cells in vitro, the differentiation efficiency of the muscle stem cells is extremely significantly improved, the differentiation potential is stimulated, more muscle-derived proteins are produced, more multinuclear myotubes can be formed in a shorter time, and thicker and longer muscle fibers are formed. The chemically defined muscle stem cell differentiation medium can also provide higher differentiation efficiency when producing cell cultured meat in 3D, express more myosin heavy chains, and produce cell cultured meat with higher quality.

Compared with the existing cell differentiation medium, using the chemically defined modified cell differentiation medium as described in the present invention can increase the relative expression of MYOG genes by 4.48 times on the 2^{nd} day of differentiation, increase the relative expression of MYHC genes by 55.28 times on the 6^{th} day of differentiation, express more myosin heavy chains (5% more) on the 5^{th} day of differentiation, and increase the percentage of cell differentiation from 34.94% to 57.93% in the terminal differentiation stage, so the difference is extremely significant, and more, thicker and longer muscle fibers are formed through induced differentiation. The above improvement of the differentiation performance provides a more efficient and inexpensive method for differentiation of muscle stem cells into myotubes, and for 3D culture of muscle stem cells to produce cell cultured meat.

### BRIEF DESCRIPTION OF THE DRAWINGS

FIG. 1: The chemically defined modified cell differentiation medium added with cell culture cofactors induces differentiation of muscle stem cells on the 5^{th} day.

FIG. 2: The existing cell differentiation medium containing 2% horse serum induces differentiation of muscle stem cells on the 5^{th} day (positive control).

FIG. 3: Comparison of the chemically defined modified cell differentiation medium added with cell culture cofactors and the existing cell differentiation medium containing 2% horse serum (positive control) in induction of differentiation of muscle stem cells on the 5^{th} day (by MYHC/DAPI staining).

FIG. 4: The expression of MYOG genes in pre-differentiation and on the 2^{nd}, 4^{th} and 6^{th} days of differentiation using the chemically defined modified cell differentiation medium added with cell culture cofactors and the existing cell differentiation medium containing 2% horse serum respectively to induce the differentiation of muscle stem cells.

FIG. 5: The expression of MYHC genes in pre-differentiation and on the 2^{nd}, 4^{th} and 6^{th} days of differentiation using the chemically defined modified cell differentiation medium added with cell culture cofactors and the existing cell differentiation medium containing 2% horse serum respectively to induce the differentiation of muscle stem cells.

FIG. 6: The expression of CAV-3 genes in pre-differentiation and on the 2nd, 4^{th} and 6th days of differentiation using the chemically defined modified cell differentiation medium added with cell culture cofactors and the existing cell differentiation medium containing 2% horse serum respectively to induce the differentiation of muscle stem cells.

FIG. 7: Experimental results of Western blotting (MYHC protein) in pre-differentiation and on the 1^{st} and 5^{th} days of differentiation using the chemically defined modified cell differentiation medium added with cell culture cofactors and the existing cell differentiation medium containing 2% horse serum respectively to induce the differentiation of muscle stem cells.

FIG. 8: Comparison of the percentage of cell differentiation on the 5^{th} day of induction of differentiation of muscle stem cells using the chemically defined muscle stem cell differentiation medium added with cell culture supplement factors and the existing differentiation medium containing 2% horse serum.

### DETAILED DESCRIPTION

The chemically defined cell differentiation medium provided by the present invention is a cell medium added with cell culture supplement factors, and other aspects are consistent with a normal in vitro culture method of muscle stem cells.

The formula of a muscle stem cell proliferation medium used in the proliferation stage in the following embodiments includes 79 vol % of F10 basal medium, 20 vol % of fetal bovine serum, 1 vol % of penicillin-streptomycin double antibody, and 5 ng/ml recombinant human fibroblast growth factor (Basic Fibroblast Growth Factor, bFGF).

The existing muscle stem cell differentiation medium (positive control) used in the following embodiments is a cell medium containing 97 vol % of DMEM/F12 basal medium, 2 vol % of horse serum and 1 vol % of penicillin-streptomycin double antibody.

The chemically defined modified cell differentiation medium used in differentiation of muscle stem cells in vitro used in the following embodiments contains 99 vol % of DMEM/F12 basal medium with 1 vol % of penicillin-streptomycin double antibody added, and supplemented with cell culture cofactors as shown in Table 1.

TABLE 1

| Component | Name | Concentration | |
|---|---|---|---|
| Component A | N-2-hydroxyethylpiperazine-N-2- | 1.2 | mg/mL |
| | ethanesulfonic acid | | |
| Component B | PEG-PPG-PEG | 0.9 | mg/mL |
| Component C | Serum albumin | 0.5 | mg/mL |
| Component D | Tween | 0.1 | mg/mL |
| Component E | Transferrin | 2 | μg/mL |
| Component F | Ethanolamine | 2 | μg/mL |
| Component G | Cholesterol | 1 | μg/mL |
| Component H | Vitamin E | 1 | μg/mL |
| Component I | Palmitic acid | 0.5 | μg/mL |
| Component J | Stearic acid | 0.3 | μg/mL |
| Component K | Insulin | 0.1 | μg/mL |
| Component L | Palmitoleic acid | 0.1 | μg/mL |
| Component M | Linolenic acid | 0.1 | μg/mL |
| Component N | Arachidonic acid | 0.08 | μg/mL |
| Component O | Myristic acid | 0.05 | μg/mL |
| Component P | Oleic acid | 0.05 | μg/mL |
| Component Q | Sodium selenite | 0.05 | μg/mL |
| Component R | Linoleic acid | 0.05 | μg/mL |
| Component S | IGF | 0.05 | μg/mL |

The cells used in the following embodiments are muscle stem cells from young pigs, further, adherent cells, and still further, muscle stem cells with a positive rate of Pax7 surface antibody 90% or more after being sorted by a flow cytometer.

The culture condition in the following embodiments is 37° C. in a CO₂ incubator, and the concentration of CO₂ is 5% (v/v).

The detection methods used in the following embodiments, unless otherwise specified, are experimental methods, detection methods and preparation methods disclosed in the field. For details, see Shijie Ding et al., “Maintaining bovine satellite cells stemness through p38 pathway” (DOI: 10.1038/s41598-018-28746-7).

The materials, reagents and the like used in the following embodiments are commercially available unless otherwise specified.

### Embodiment 1 Induced Differentiation of Pig Muscle Stem Cells

The experiment includes two groups, namely a group (positive control) treated with the existing cell differentiation medium, and a group treated with the chemically defined modified cell differentiation medium as described in the present invention. The specific treatment methods are as follows:

1) Matrigel plating: A solution of Matrigel and PBS in a ratio of 1:50 (Val) was prepared and added to a 3.5 cm culture dish as per 1 mL/plate. The culture dish was placed in a CO₂ incubator for 1-6 h, and then taken out, blotted dry, washed 2 times with PBS, and blotted dry again.

2) Cell inoculation: High-purity pig muscle stem cells before the P6 generation were inoculated into the 3.5 cm culture dish at a density of 60000-120000 cells/plate after Matrigel plating. The cells were cultured in a proliferation medium added with 5 ng/ml recombinant human fibroblast growth factor (bFGF), and the medium was changed every two days.

3) Differentiation induction: After the cells proliferated and divided to cover the entire culture dish (muscle stem cells expanded to a density of 90% or more), the muscle stem cell proliferation medium was blotted. The cells were washed with the existing muscle stem cell differentiation medium (positive control) and the chemically defined modified cell differentiation medium as described in the present invention, respectively. The corresponding differentiation medium was added at a volume of 2 mL/plate to induce differentiation, half of the medium was exchanged every 2 days, and the effect of differentiation after muscle fibers are formed by differentiation was observed on the 5^{th} day.

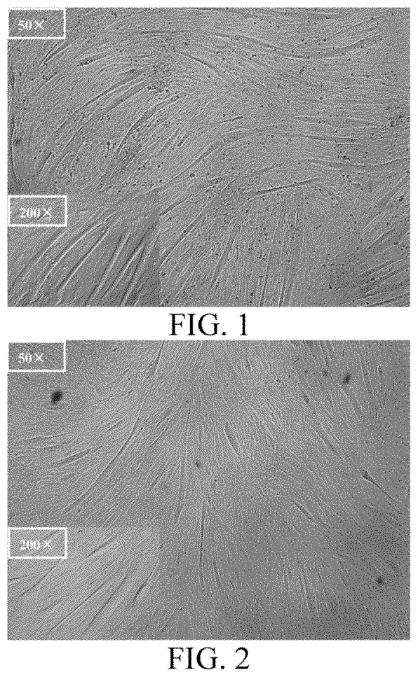

4) The results show that compared with the conventional muscle stem cell differentiation medium, the modified cell differentiation medium provided by the present invention can promote the differentiation ability of the muscle stem cells in vitro, has higher differentiation efficiency, and forms more, longer and thicker myotubes (FIG. 1 and FIG. 2).

### Embodiment 2 Immunofluorescence Detection in Induced Differentiation of Pig Muscle Stem Cells

1) Sample collection: After 5 days of induced differentiation, the myotube matured cells in the group treated with the existing muscle stem cell differentiation medium (positive control) and the group treated with the chemically defined modified muscle stem cell differentiation medium as described in the present invention in Embodiment 1 were collected, the medium was blotted, the cells were washed 1 time with PBS, and 4% (m/V) paraformaldehyde (percent weight in volume) was added for fixation at 4° C. overnight.

2) MYHC protein and DAPI immunofluorescence staining: 4% paraformaldehyde was blotted. The cells were washed with PBS 3 times while shaking for 5 min each time with a shaker, circles were drawn with a Pap pen in the period, and about 50 μL of 0.5% trizol was added to each circle. The cells were shaken for 15 min and then washed 3 times with PBS, MYHC antibody (1:800) diluted with 1% BSA was added, and the cells were incubated at 4° C. for 16 h and washed 3 times with PBS to remove the antibody. The secondary antibody (1:500) diluted with 1% BSA was added, and the cells were incubated in the dark for 1 h and washed once with PBS. A DAPI mounting medium was added and covered with a cover glass, and a photo was taken (FIG. 3).

3) Statistics of cell differentiation rate: The number of nuclei was counted using Image J software, the nuclei with a size greater than 50 square inches inside/outside the myotubes and the roundness between 0 and 1 were counted, and counting and significance analysis were performed (FIG. 8).

4) The results showed that the percentage of cell differentiation in the group treated with the existing muscle stem cell differentiation medium (positive control) was 34.94%±2.7% (n=6), and the percentage of cell differentiation in the group treated with the chemically defined modified cell differentiation medium as described in the present invention was 57.93%±3.96% (n=6). It can be seen that the chemically defined modified cell differentiation medium can extremely significantly (P<0.0001) increase the percentage of cell differentiation, allowing more cells to participate in fusion to form muscle fibers.

### Embodiment 3 Gene and Protein Level Detection in Induced Differentiation of Pig Muscle Stem Cells

1) Gene level detection:

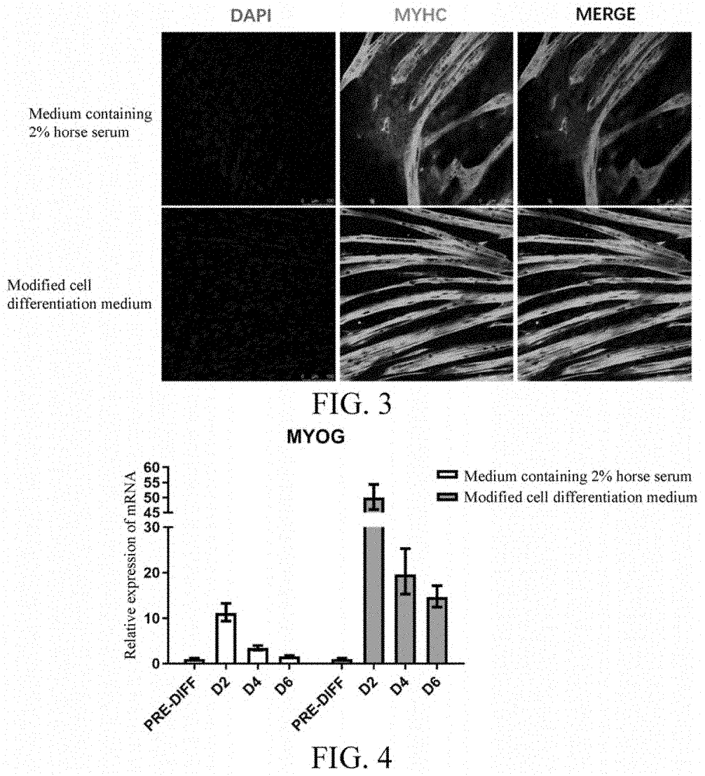

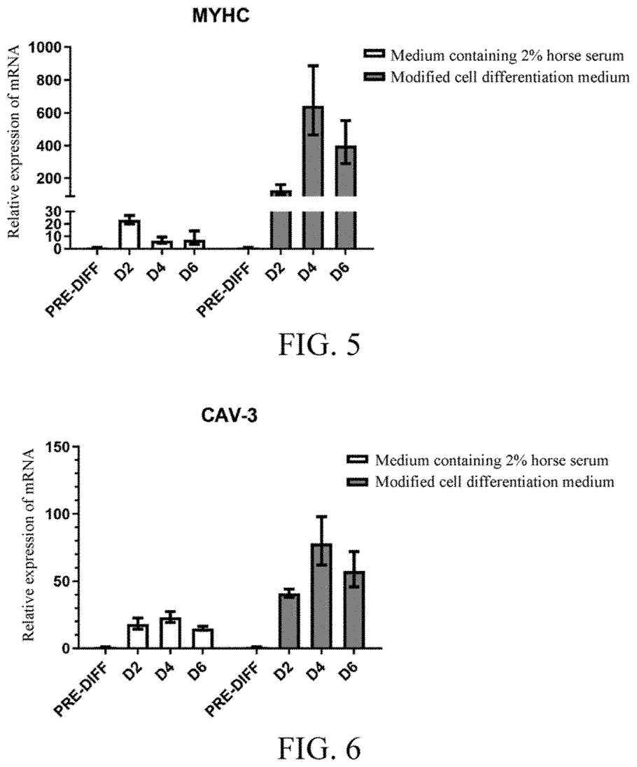

According to the treatment method of Embodiment 1, samples were taken on the 2^{nd}, 4^{th}, and 6^{th} days of induced differentiation in step 3), and the gene expressions of MYOG, MYHC, and CAV-3 on the 2^{nd}, 4^{th}, and 6^{th} days of culture in the existing muscle stem cell differentiation medium (positive control) and the chemically defined modified cell differentiation medium as described in the present invention were detected by real-time fluorescence quantitative PCR (FIG. 4, FIG. 5 and FIG. 6), MYOG genes were generally highly expressed in the early stage of differentiation, indicating the differentiation ability of muscle stem cells, and the expression of MYHC increased continuously in the differentiation progress, indicating the differentiation level of muscle stem cells.

The results show that compared with the group treated with the existing muscle stem cell differentiation medium (positive control), the group treated with the chemically defined modified cell differentiation medium as described in the present invention could increase the relative expression of MYOG genes by 4.48 times on the 2^{nd} day of differentiation, and increase the relative expression of MYHC genes by 55.28 times on the 6^{th} day of differentiation. Therefore, the chemically defined modified cell differentiation medium as described in the present invention improves the differentiation ability and differentiation level of muscle stem cells.

2) Protein level:

According to the treatment method of Embodiment 1, samples were taken on the 1^{st} and 5^{th} day of induced differentiation in step 3). 2 plates of cells cultured by the existing muscle stem cell differentiation medium (positive control) and 2 plates of cells cultured by the chemically defined modified cell differentiation medium as described in the present invention were taken on the 1^{st} and 5^{th} day respectively. 100 μL of radio immunoprecipitation assay (RIPA) lysis buffer (added with (phenylmethanesulfonyl fluoride) PMSF with a final concentration of 1 mM) was added to each plate of cells, and the cells were lysed on ice for 30 min, collected and stored at −20° C. for use. After centrifugation at 12000 g for 5 min, the supernatant was collected, and the protein concentration was determined using a BCA kit of Thermo Fisher. 5× loading buffer was added at 4:1 (V:V), mixed well, heated at 95° C. for 5 min to denature the protein, and stored at −80° C.

SDS-page gel electrophoresis: A running buffer and a blotting buffer were prepared in advance (10% methanol was additionally added to the blotting buffer). Under the condition that the running buffer is 12% higher than a denaturing agarose prefabricated gel plate, 20 ug of denatured protein was added to loading wells respectively. The voltage was set at 80 V for 30 min and 120 V for 90 min for performing electrophoresis and the above two procedures, and whether a protein loading buffer reaches the bottom of the prefabricated plate was observed.

Membrane transfer: A polyvinylidene fluoride (PVDF) membrane was activated in methanol for about 10 s, put into a blotting buffer and stored for use. Sponge, 2 layers of filter paper, gel, the activated PVDF membrane, 2 layers of filter paper and sponge were placed, clamped with a holder, and put in an electrophoresis tank, and the prepared blotting buffer was added at 90 V and run for 90 min.

Blocking: The PVDF membrane after the transfer was put into a blocking buffer (5% skim milk powder prepared with TBST), and blocked in a shaker at room temperature for 2 h, and then the blocking buffer was blotted.

Primary and secondary antibody incubation: A MyHC primary antibody was diluted according to the specific conditions of the antibody and incubated at 4° C. for 14-16 h. After the primary antibody incubation, the primary antibody was recovered and the cells were washed with TBST three times for 5 min each. A diluted secondary antibody was added and incubated for 2 h, and then the cells were washed with TBST three times for 5 min each.

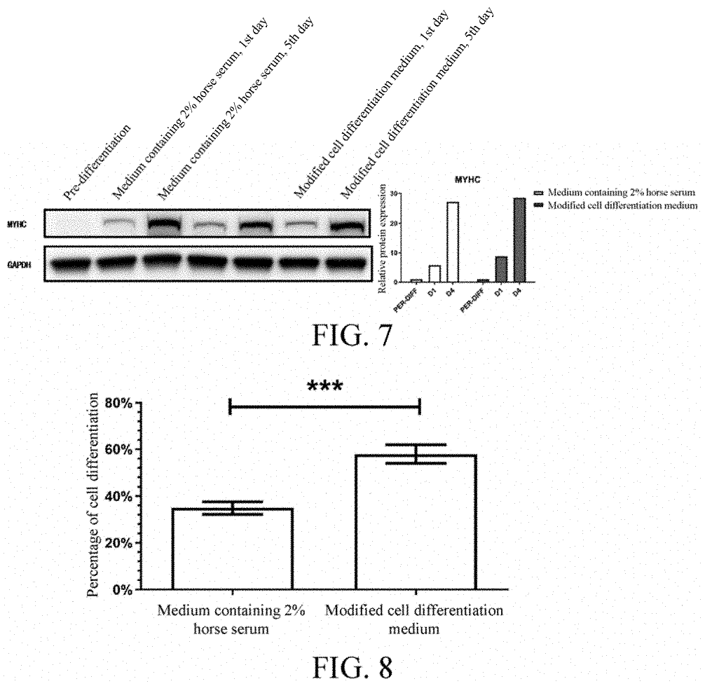

Development: The PVDF membrane was covered with a developer in the dark and incubated for 5 min, the developer was blotted, and a photo was taken under a gel imager. Grayscale analysis was performed using Quantity One analysis software. The internal reference protein used in the experiment was Gapdh (FIG. 7).

The results showed that the group treated with the chemically defined modified cell differentiation medium as described in the present invention could increase the expression of intracellular myosin heavy chains by 28.5 times within 5 days, and compared with the group treated with the existing cell differentiation medium containing 2% horse serum (positive control), the modified cell differentiation medium as described in the present invention had a higher myosin weight content (5% higher) on the 5^{th} day of induced differentiation.

### Embodiment 4 Preparation of Cultured Meat

1) Type I collagen, a DMEM containing phenol red, and a sodium hydroxide solution were mixed to obtain a mixed solution. The volume ratio of the type I collagen to the DMEM containing phenol red is 50:40, and the pH value is adjusted to 7.3-7.5 by adding the sodium hydroxide solution. Matrigel was added to the mixed solution, and the volume ratio of the Matrigel to the mixed solution was 8:91.5.

2) The muscle stem cells were mixed with the mixed solution obtained in step 1), and a cell-containing mixed solution with a density of 1×10⁶ cells/ml was obtained.

3) The cell-containing mixed solution obtained in step 2) was divided into two groups, added to a porous reticular production mold of cultured meat as described in the patent with the number of ZL201921875316.X respectively, and cultured in a 5% carbon dioxide incubator at 37° C. for 2 h to form hydrogel muscle tissues. After a proliferation medium was added to the hydrogel muscle tissues for 1 day, the proliferation medium in one group was replaced with the chemically defined modified cell differentiation medium, the proliferation medium in the other group was replaced with the existing cell differentiation medium, and the hydrogel muscle tissues were cultured in the two media respectively. After culturing in the porous reticular production molds of cultured meat for 5 days, porous reticular muscle tissues were obtained, namely cultured meat products.

The results showed that compared with the existing cell differentiation medium containing 2% horse serum, the group treated with the chemically defined modified cell differentiation medium as described in the present invention had earlier tissue contraction during the preparation of cell cultured meat, the resulting cultured meat had the advantage of having more, longer, thicker, and regularly oriented muscle fibers, and more myosin heavy chains were produced.

The embodiments disclosed above are intended to describe the implementation cases disclosed by the present invention, but should not be construed as limitations of the present invention. Many different cell culture cofactors are listed herein, which may be used in further combinations, without departing from the scope and spirit of the present invention, so that the present invention is not limited to the disclosed implementation cases. In fact, various modifications apparent to those skilled in the art that are made to obtain the present invention should fall within the scope of the present invention.

What is claimed is:

1. A chemically defined modified cell differentiation medium for differentiation of muscle stem cells in vitro, wherein the modified cell differentiation medium is a muscle stem cell differentiation medium added with cell culture cofactors, and does not contain serum components.

2. The modified cell differentiation medium according to claim 1, wherein the muscle stem cell differentiation medium comprises a muscle stem cell basal medium and a penicillin-streptomycin double antibody solution, and the volume ratio of the muscle stem cell basal medium to the penicillin-streptomycin double antibody solution is 99:1;
preferably, the muscle stem cell basal medium is one selected from DMEM, MEM, DMEM/F12, and F10; and
preferably, in the penicillin-streptomycin double antibody solution, the content of penicillin is 10000 U/ml, and the content of streptomycin is 10 mg/ml.

3. The modified cell differentiation medium according to claim 1, wherein the cell culture cofactors are several selected from the following cell culture supplement factors: N-2-hydroxyethylpiperazine-N-2-ethanesulfonic acid, PEG-PPG-PEG, serum albumin, Tween, transferrin, ethanolamine, cholesterol, vitamin E, palmitic acid, stearic acid, insulin, palmitoleic acid, linolenic acid, arachidonic acid, myristic acid, oleic acid, sodium selenite, linoleic acid, and insulin-like growth factor (IGF).

4. The modified cell differentiation medium according to claim 1, wherein in the modified cell differentiation medium, the total concentration of cell culture cofactors added is in a range of 0.05-50 mg/mL;
preferably, in the modified cell differentiation medium, the total concentration of the cell culture cofactors added is in a range of 0.5-30 mg/mL; and
further preferably, in the modified cell differentiation medium, the total concentration of the cell culture cofactors added is 3 mg/mL.

5. The modified cell differentiation medium according to claim 3, wherein in the modified cell differentiation medium, the concentration of any cell culture cofactor added is in a range of 0.5 ng/mL-100 mg/ml;
preferably, in the modified cell differentiation medium, the concentration of any cell culture cofactor added is in a range of 50 ng/mL-50 mg/ml; and
further preferably, in the modified cell differentiation medium, the concentration of any cell culture cofactor added is in a range of 50 ng/mL-1.2 mg/ml.

6. An application of the modified cell differentiation medium according to claim 1 in inducing differentiation of muscle stem cells in vitro, wherein the modified cell differentiation medium according to claim 1 is used to induce differentiation of muscle stem cells in vitro.

7. The application according to claim 6, wherein the modified cell differentiation medium can promote differentiation of muscle stem cells in a process of inducing differentiation in vitro.

8. The application according to claim 6, wherein the modified cell differentiation medium can increase the expressions of myogenin (MYOG), myosin heavy chain (MYHC) and caveolin-3 (CAV-3) genes in the process of inducing differentiation of muscle stem cells in vitro.

9. An application of the modified cell differentiation medium according to claim 1 in preparation of cultured meat, wherein the application comprises the following steps:
1) mixing type I collagen, a DMEM containing phenol red, and a sodium hydroxide solution to obtain a mixed solution;
2) mixing muscle-derived cells with the mixed solution obtained in step 1) to obtain a cell-containing mixed solution; and
3) adding the cell-containing mixed solution obtained in step 2) to a porous reticular production mold of cultured meat for performing culture; performing culture in a 5% carbon dioxide incubator at 37° C. for 2 h to form hydrogel muscle tissues; after adding a proliferation medium to the hydrogel muscle tissues for 1 day, replacing the proliferation medium with the chemically defined modified cell differentiation medium according to claim 1; culturing the hydrogel muscle tissues in the medium, and performing culture in the porous reticular production mold of cultured meat for 5 days to obtain porous reticular muscle tissues;
preferably, the volume ratio of the type I collagen to the DMEM containing phenol red in step 1) is 50:40, and the pH value is adjusted to 7.3-7.5 by adding the sodium hydroxide solution;
preferably, step 1) further comprises adding Matrigel to the mixed solution, wherein the volume ratio of the Matrigel to the mixed solution is 8:91.5, the Matrigel and the mixed solution are uniformly mixed and added to the mold for culture, and the Matrigel can help the reticular muscle tissue differentiation; and
preferably, the muscle-derived cells in step 2) are one of muscle stem cells, muscle progenitor cells, and muscle precursor cells, and in the cell-containing mixed solution, the density of the muscle-derived cells is 1×10⁵ cells/ml−1×10⁷ cells/ml.

10. Cultured meat obtained by the modified cell differentiation medium according to claim 1.
